(19) United States
(12) Patent Application Publication
(10) Pub. No.: US 2014/0128779 A1
(43) Pub. Date: May 8, 2014

(54) MEDICAL DEVICE FOR REMOVING PATHOGENS

(71) Applicant: Sheldon Gibson, Nedrow, NY (US)

(72) Inventor: Sheldon Gibson, Nedrow, NY (US)

(21) Appl. No.: 14/073,289

(22) Filed: Nov. 6, 2013

Related U.S. Application Data
(60) Provisional application No. 61/723,144, filed on Nov. 6, 2012.

(51) Int. Cl. A61N 7/00

(52) U.S. Cl. CPC A61N 7/00
USPC 601/2

(57) ABSTRACT

A medical device for removing pathogens from a patient is disclosed. The medical device comprises a signal generator that produces a sound wave transmitted from a single electrode into the body of the patient. The single electrode disc can be disposed on the surface of an ergonomically designed housing that can be held by the patient in one hand or otherwise rest against the surface of the body of the patient hands-free.

### CROSS-REFERENCE TO RELATED APPLICATIONS

This application claims the benefit of and priority to U.S. Provisional Patent Application Ser. No. 61/723,144, filed Nov. 6, 2013, and entitled “MEDICAL DEVICE FOR REMOVING PATHOGENS,” the entirety of which is incorporated herein by reference.

### BACKGROUND OF THE INVENTION

The subject matter disclosed herein relates to a medical device for removing pathogens from a patient.

Certain devices are currently used for removing pathogens (e.g., viruses, bacteria, parasites, toxins, microorganisms, molds, etc.) from patients using high frequency signals (e.g., 30 kHz) generated by a signal generator having two electrodes. One exemplary device is the ZAPPER developed by Hulda R. Clark. With the ZAPPER or similar devices, a patient is required to hold the first electrode in one hand (or attach the first electrode to a first body part) and hold the second electrode in the other hand (or attach the second electrode to a second body part). The high frequency oscillating current travels through the patient's body between the two electrodes killing the pathogens in the body that it encounters.

In many cases, the treatment can last several minutes (e.g., ten or twenty minutes) and may need to be repeated several times a day. The requirement that a patient hold the two electrodes, occupying both of the patient's hands typically prevents the patient from conducting other activities (e.g., eating, drinking, driving, operating a phone, operating a television remote, etc.). It would be advantageous to design a medical device that provides treatment not requiring the use of two hands, allowing a patient to conduct other activities during treatment or to be more relaxed.

The discussion above is merely provided for general background information and is not intended to be used as an aid in determining the scope of the claimed subject matter.

### BRIEF DESCRIPTION OF THE INVENTION

A medical device for removing pathogens from a patient is disclosed. The medical device comprises a signal generator that produces a sound wave transmitted from a single electrode into the body of the patient. The single electrode disc can be disposed on the surface of an ergonomically designed housing that can be held by the patient in one hand or otherwise rest against the surface of the body of the patient hands-free. An advantage that may be realized in the practice of some of the disclosed embodiments of the medical device is that it allows the patient to perform other activities during treatment or to simply be more relaxed during the treatment.

In one embodiment, a device for removing pathogens from a body is disclosed. The device includes a housing, a signal generator disposed inside of the housing, wherein the signal generator is configured to produce a sound wave, and a single electrode disposed on a surface of the housing and coupled to the signal generator, wherein the single electrode is configured to contact the body and transmit the sound wave into the body to remove pathogens from the body without the use of a second electrode.

In another embodiment, a method for removing pathogens from a body with a medical device is disclosed. The method includes the steps of placing only a single electrode disposed on the surface of the medical device in contact with the body, producing a sound wave, and transmitting the sound wave into the body through only a single electrode to remove pathogens from the body without the use of a second electrode.

This brief description of the invention is intended only to provide a brief overview of subject matter disclosed herein according to one or more illustrative embodiments, and does not serve as a guide to interpreting the claims or to define or limit the scope of the invention. This brief description is provided to introduce an illustrative selection of concepts in a simplified form that are further described below in the detailed description. This brief description is not intended to identify key features or essential features of the invention, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic diagram of an exemplary medical device;

FIG. 2 is a perspective view of an exemplary spherical-shaped medical device;

FIG. 3A is a top view of an exemplary disc-shaped medical device;

FIG. 3B is a side view of the exemplary disc-shaped medical device of FIG. 3A;

FIG. 4A is a side view of an exemplary stick-shaped medical device; and

FIG. 4B is a top view of the exemplary stick-shaped medical device of FIG. 4A.

### DETAILED DESCRIPTION OF THE INVENTION

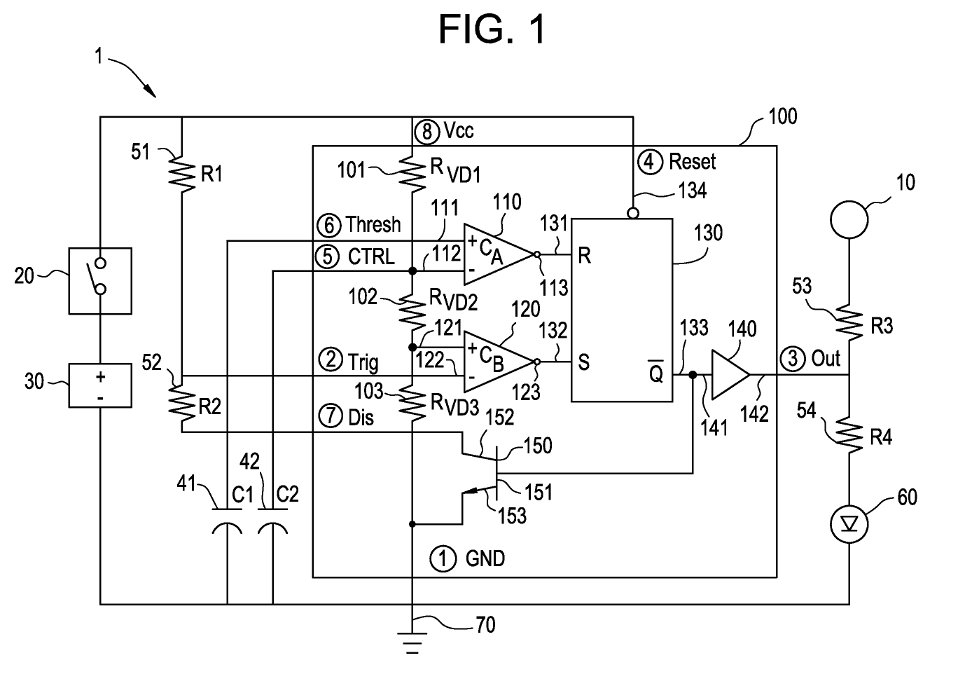

FIG. 1 is a schematic diagram of an exemplary medical device 1. The medical device 1 includes an integrated circuit (IC) signal generator 100. In different embodiments, the IC signal generator 100 can be a 555 Timer, 7555 Timer, CMOS TLC555 Timer, or other similar timers, pulse generators, or oscillators that can generate simple pulses or continuous waves (square waves, sine waves, etc.). The type of signal produced by the IC signal generator 100 is determined by the types and configuration of external components connected to the inputs and outputs of the IC signal generator 100 (e.g., input 1 (ground), input 2 (trigger), output 3, input 4 (reset), input 5 (voltage control), input 6 (threshold), output 7 (discharge), and input 8 (voltage supply (V_{CC})).

The IC signal generator 100 can include two voltage comparators, voltage comparator C_{A} 110 and voltage comparator C_{B} 120. Each comparator has a noninverting (+) input and an inverting (−) input. Whenever the voltage at the noninverting (+) input is more positive than the voltage at the inverting (−) input, a positive voltage is produced at the comparator output. Whenever the voltage at the inverting (−) input is equal to or greater than the voltage at the noninverting (+) input, zero (0) volts are produced at the comparator output. In other words, when the voltage at the noninverting (+) input is higher than the voltage at the inverting (−) input, the output of the comparator is positive, while the output of the comparator is zero (0) under all other conditions.

The voltage comparators C_{A} 110, C_{B} 120 work in conjunction with a voltage divider network formed by voltage divider resistors R_{VD1} 101, R_{VD2} 102, and R_{VD3} 103 connected in series between the voltage (DC) supply 30 (e.g., solar cell, battery of 9V DC, rechargeable battery, etc.) (through switch 20) and ground 70. The switch 20 can be, e.g., a toggle switch, slide switch, micro mini Light Emitting Diode (LED) switch, etc. used for connecting and disconnecting the voltage supply 30. In one embodiment, each voltage divider resistor R_{VD1} 101, R_{VD2} 102, R_{VD3} 103 has the same resistance (e.g., 5 k ohms) so that one-third of the voltage supply (e.g., 3V) drops across each resistor. Accordingly, two-thirds of the voltage supply 30 (e.g., 6V) is applied at the inverting (−) input 112 of comparator C_{A} 110, while one-third of the voltage supply 30 (e.g., 3V) is applied at the noninverting (+) input 121 of comparator C_{B} 120. The non-inverting (+) input 111 of comparator C_{A} 110 is connected to capacitor C₁ 41 (e.g., 0.01 μF), which is connected to ground 70 forming the threshold voltage for comparator C_{A} 110. The inverting (−) input 112 of comparator C_{A} 110 is connected to capacitor C₂ 42 (e.g., 0.0047 μF), which is connected to ground 70 forming the control voltage for comparator C_{A} 110. It should be noted that the values of the capacitors C₁ 41, C₂ 42 in the exemplary medical device 1 are reversed from the values used in the existing ZAPPER devices.

The output 113 of comparator C_{A} 110 is connected to the R input 131 of the RS Flip-Flop 130, while the output 123 of comparator C_{B} 120 is connected to the S input 132 of the RS Flip-Flop 130. The Q̅ output 133 of the RS Flip-Flop 130 is connected to the input 141 of the output buffer 140 and the base 151 of the discharge transistor (NPN) 150. Whenever the output 113 of comparator C_{A} 110 is positive, the Q̅ output 133 of the RS Flip-Flop 130 becomes positive also (logic HIGH). Whenever the output 123 of comparator C_{B} 120 is positive, the Q̅ output 133 of the RS Flip-Flop 130 becomes zero (logic LOW). Whenever the outputs 113, 123 of both comparators C_{A} 110, C_{B} 120 are zero, the Q̅ output 133 of the RS Flip-Flop 130 remains unchanged. The outputs 113, 123 of both comparators C_{A} 110, C_{B} 120 are never positive at the same time. The R̅E̅S̅E̅T̅ input 134 of the RS Flip-Flop 130 is connected to the voltage supply 30 (through switch 20).

The discharge transistor (NPN) 150 acts like a switch. When a positive voltage is present at the base 151 (which is connected to the Q̅ output 133 of the RS Flip-Flop 130), the discharge transistor (NPN) 150 turns on and passes current between the emitter 152 and the collector 153. When zero (0) volts are present at the base 151 (which is connected to the Q̅ output 133 of the RS Flip-Flop 130), the discharge transistor (NPN) 150 turns off and blocks current between the emitter 152 and the collector 153. The collector 153 of the discharge transistor (NPN) 150 is connected to ground 70. A resistor R₂ 52 (e.g., 3.9 k ohms) is connected between the emitter 152 of the discharge transistor (NPN) 150 and the inverting (−) input 122 of comparator C_{B} 120. A resistor R₁ 51 (e.g., 1 k ohms) is connected between the inverting (−) input 122 of comparator C_{B} 120 and the voltage supply 30 (through switch 20).

The output buffer 140 produces a high current voltage, which provides a sufficient output 142 to power external circuitry. The input 141 of the output buffer 140 is connected to the Q̅ output 133 of the RS Flip-Flop 130. The output 142 of the output buffer 140 goes positive when the Q̅ output 133 of the RS Flip-Flop 130 is zero (logic LOW). The output 142 of the output buffer 140 approaches zero when the Q̅ output 133 of the RS Flip-Flop 130 is positive (logic HIGH). A resistor R₃ 53 (e.g., 1 k ohms) is connected between a transmitting disc 10 (e.g., an electrode) and the output 142 of the output buffer 140. A resistor R₄ 54 (e.g., 3.9 k ohms) is connected between an LED 60 and the output 142 of the output buffer 140. The LED 60 is connected between resistor R₄ 54 and ground 70 and is illuminated when the sound wave is generated.

The signal generator 100 of the exemplary medical device 1 illustrated in FIG. 1 can produce a sound wave (e.g., with a frequency of 20 kHz or less to be heard by humans which can be adjusted based on the values and configuration of the resistors and capacitors connected to the IC signal generator 110). The signal generator 100 is coupled to the transmitting disc 10 so that the sound wave can be transmitted into a patient in contact with the transmitting disc 10. The transmitting disc 10 can be made of various conductive materials, including silver (e.g., 92.5) or gold (e.g., 14 k/18 k) that have antibacterial properties. The coloring of the transmitting disc 10 may be altered during treatment based on the extent of the pathogens found in the patient. Since the exemplary medical device 1 relies upon the transmission of a sound wave into a patient for the treatment rather than electrical current traveling through the patient (which required contact with two electrodes), the medical device 1 only needs a single transmitting disc 10, which can be provided in a housing that can easily be held in one hand or otherwise rest against the body of a patient.

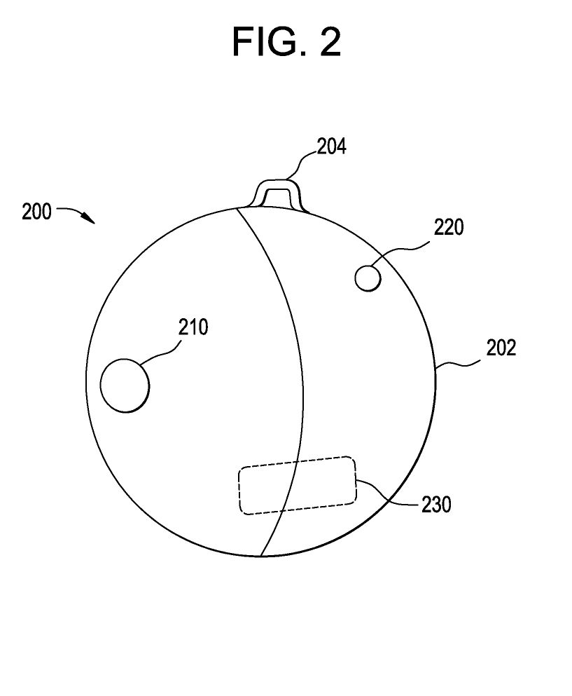

FIGS. 2 through 4B show different shapes and sizes of the housings 202, 302, 402 for different embodiments of medical devices 200, 300, 400. It will be understood that the medical device 1 of FIG. 1 can be embodied in housings 202, 302, 402 of various shapes and sizes, not limited to those shown in the figures. The housings 202, 302, 402 can be fabricated with high impact plastic forming a waterproof enclosure. In some embodiments, the housings 202, 302, 402 can be fabricated with transparent (clear or colored) resins. In some embodiments, bails 204, 304, 404 can be provided on the surfaces of the housings 202, 302, 402 for use with a lanyard, strap, or other device to secure the medical devices 200, 300, 400. The bails 204, 304, 404 can be sized to accommodate relatively small devices such as rings or bracelets. In addition, while the exemplary medical devices 200, 300, 400 are shown with batteries 230, 430 or solar cells 330 as the voltage supplies 30 (FIG. 1), it will be understood that other voltages supplies can be used if available. If solar cells are used, it may be unnecessary to include switches 220, 320, 420.

FIG. 2 is a perspective view of an exemplary spherical-shaped medical device 200. The spherical-shaped medical device 200 includes a housing 202, bail 204, transmitting disc 210, switch 220, and battery (e.g., 9V) 230. As can be seen in FIG. 2, the housing 202 can be sized (e.g., a diameter in the range of 50 mm to 90 mm, with other sizes as well) to be held in a patient's hand with the transmitting disc 210 contacting the palm or other portion of the hand during treatment. At least a portion of the transmitting disc 210 and switch 220 are disposed on the surface of the housing 202, while the battery 230 is enclosed within the housing 202. The transmitting disc 210 and the switch 220 can extend through openings in the housing 202. The housing 202 can be formed of two hemispheres that are in snap engagement with each other. The battery 230 can be held in place on a plate within the spherical-shaped medical device 200 (e.g., the bottom side of a printed circuit board supporting the electronics of FIG. 1). The plate can include arms extending from the plate to receive and secure the battery 230.

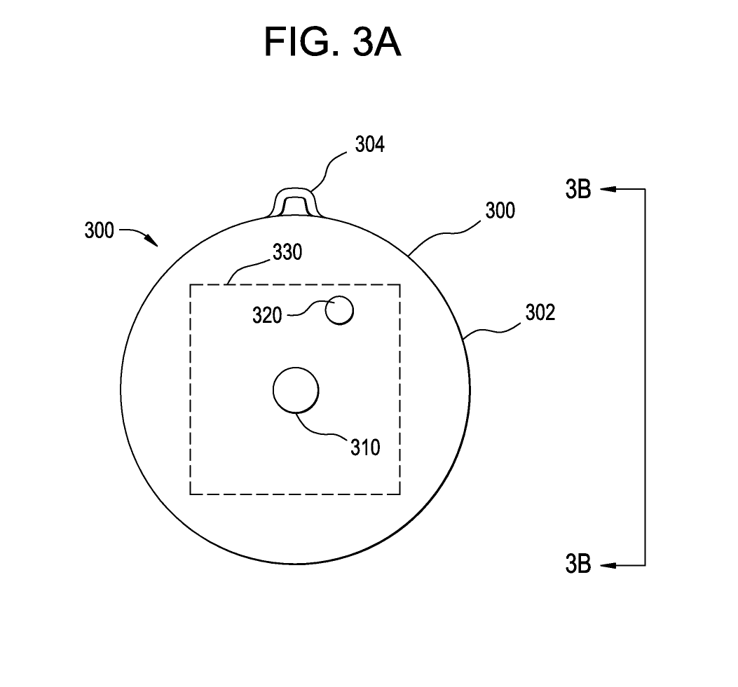

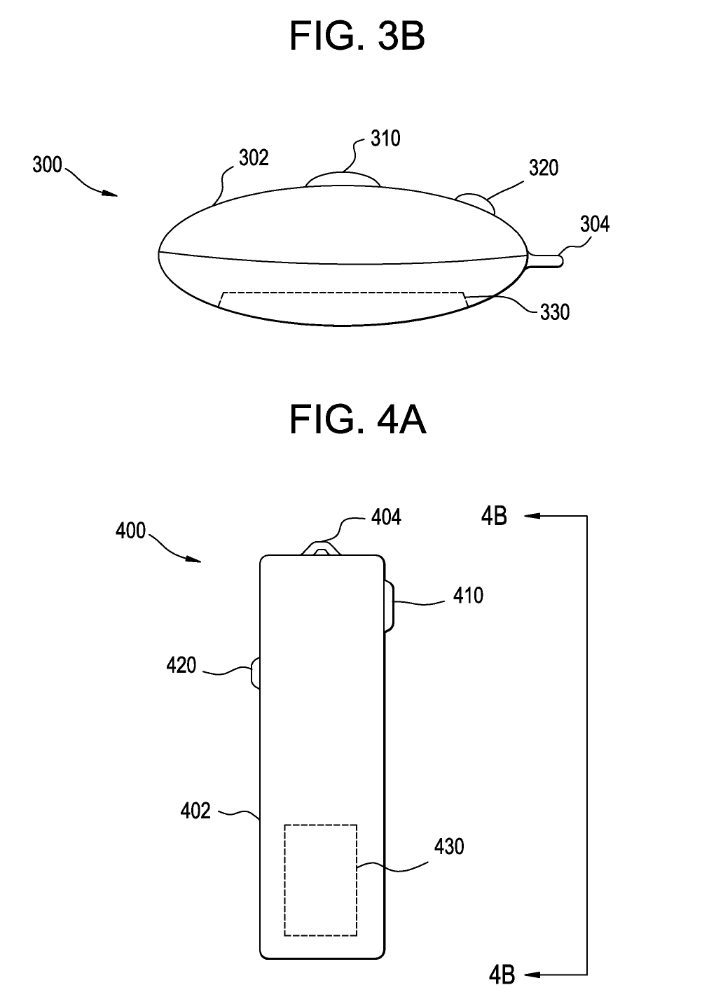

FIG. 3A is a top view of an exemplary disc-shaped medical device 300. FIG. 3B is a side view of the exemplary disc-shaped medical device 300 of FIG. 3A. The disc-shaped medical device 300 includes a housing 302, bail 304, transmitting disc 310, switch 320, and a solar cell 330. As can be seen in FIGS. 3A and 3B, the housing 302 can be sized (e.g., a diameter in the range of 50 mm to 90 mm with a height in the range of 25 mm to 45 mm, with other sizes as well) to be held in a patient's hand, with the transmitting disc 310 contacting the palm or other portion of the hand during treatment. Additionally, the disc-shaped medical device 300 can be worn as a pendant that can rest on the surface of the body of a patient hands-free, with the transmitting disc 310 contacting, e.g., the chest of the patient during treatment. In this configuration, the solar cell 330 faces away from the body of the patient and can be charged by the sun. At least a portion of the transmitting disc 310 and switch 320 are disposed on the surface of the housing 302, while the solar cell 330 (e.g., a square of 28 mm by 28 mm with rounded edges) is enclosed within the housing 302. In some embodiments, the switch 320 is not provided and the disc-shaped medical device 300 is activated when the transmitting disc 310 contacts the body of the patient. In addition, the LED 60 (FIG. 1) can be illuminated when there is sufficient power from the solar cell 330 to produce a sound wave through the transmitting disc 310 into the body of the patient.

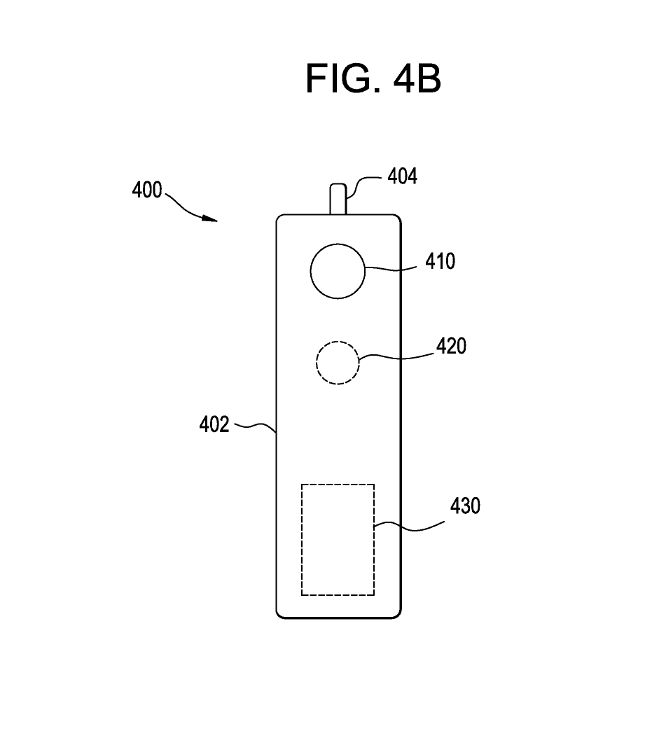

FIG. 4A is a side view of an exemplary stick-shaped medical device 400 (i.e., significantly longer than it is wide or high). FIG. 4B is a top view of the exemplary stick-shaped medical device 400 of FIG. 4A. The stick-shaped medical device 400 includes a housing 402 with rounded edges, bail 404, transmitting disc 410, switch 420, and a solar cell 430. As can be seen in FIGS. 4A and 4B, the housing 402 can be sized (e.g., length in the range of 75 mm to 150 mm, height in the range of 10 mm to 35 mm, width in the range of 10 mm to 45 mm, with other sizes as well) to be held in a patient's hand with the transmitting disc 410 contacting the palm or other portion of the hand during treatment or resting against another portion of the body (e..g, when used as a pendant). At least a portion of the transmitting disc 410 and switch 420 are disposed on the surface of the housing 402, while the battery 430 is enclosed within the housing 402. In one embodiment, the switch 420 is a push-type switch and can be incorporated with the LED 60 (FIG. 1). Although the stick-shaped medical device 400 is shown with relatively flat surfaces and edges, in other embodiments, the stick-shaped medical device 400 can have rounded and curved surfaces and edges.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A device for removing pathogens from a body, the device comprising:
a housing;
a signal generator disposed inside of the housing, wherein the signal generator is configured to produce a sound wave; and
a single electrode disposed on a surface of the housing and coupled to the signal generator, wherein the single electrode is configured to contact the body and transmit the sound wave into the body to remove pathogens from the body without the use of a second electrode.

2. The device of claim 1, further comprising a voltage supply disposed inside of the housing and coupled to the signal generator.

3. The device of claim 2, wherein the voltage supply is a battery.

4. The device of claim 2, wherein the voltage supply is a solar cell.

5. The device of claim 2, further comprising a switch disposed on the surface of the housing for connecting and disconnecting the voltage supply and the signal generator.

6. The device of claim 1, further comprising a light emitting diode configured to illuminate when the signal generator is producing a sound wave.

7. The device of claim 1, wherein the housing is spherical shaped.

8. The device of claim 7, wherein the housing comprises two hemispheres snap fit to each other.

9. The device of claim 1, wherein the housing is disc shaped.

10. The device of claim 1, wherein the housing is stick shaped.

11. The device of claim 1, wherein the single electrode is disc shaped.

12. The device of claim 1, further comprising a bail disposed on the surface of the housing to secure the device to another object.

13. The device of claim 1, wherein the sound wave is a 20 kHz square wave.

14. A method for removing pathogens from a body with a medical device, the method comprising:
placing only a single electrode disposed on the surface of the medical device in contact with the body;
producing a sound wave; and
transmitting the sound wave into the body through only a single electrode to remove pathogens from the body without the use of a second electrode.

15. The method of claim 13, wherein the step of placing only a single electrode in contact with the body is done by holding the medical device in a hand of the body.

16. The method of claim 13, wherein the step of placing only a single electrode in contact with the body is done by resting the medical device on the body without holding the medical device in a hand of the body.
